(19) United States
(12) Patent Application Publication
(10) Pub. No.: US 2023/0218139 A1
(43) Pub. Date: Jul. 13, 2023

(54) Method and Apparatus for Extending Battery Life of Capsule Endoscope

(71) Applicant: CAPSOVISION, Inc., Saratoga, CA (US)

(72) Inventors: Kang-Huai Wang, Saratoga, CA (US); Mark Hadley, Los Altos, CA (US); Chang-Wei Lin, Mountain View, CA (US); Chung-Ta Lee, Sunnyvale, CA (US)

(21) Appl. No.: 18/113,034

(22) Filed: Feb. 22, 2023

Related U.S. Application Data
(63) Continuation-in-part of application No. 17/128,142, filed on Dec. 20, 2020.
Application No. 17/128,142 is a continuation-in-part of application No. 17/383,165, filed on Jul. 22, 2021, now Pat. No. 11,612,303.

(51) Int. Cl. A61B 1/00

(52) U.S. Cl. CPC A61B 1/00004; A61B 1/00016; A61B 1/0684

(57) ABSTRACT

Method for extending battery life and a capsule endoscope using the method are disclosed. According to this method, a peak current in a current profile consumed by the capsule endoscope is identified, where the peak current is contributed by at least two sub-tasks associated with operations of the capsule endoscope and said at least two sub-tasks are performed overlapping in time. A running voltage indicating a battery output voltage at or near time instances of the peak current is determined. When the running voltage a condition caused by IR (battery internal-resistance) voltage drop, the sub-tasks are adjusted to reduce overlapping so as to reduce the peak current to a second peak current. According to another method, at least one sub-task is switched to another sub-task when the running voltage is below a threshold.

### CROSS REFERENCE TO RELATED APPLICATIONS

The present invention is a Continuation-in-Part Application of and claims priority to U.S. patent application Ser. No. 17/128,142, filed on Dec. 20, 2020 and U.S. patent application Ser. No. 17/383,165, filed on Jul. 22, 2021. The U.S. Patent Applications are hereby incorporated by reference in their entireties.

### FIELD OF THE INVENTION

The present invention relates to battery-powered devices, including capsule cameras for imaging the gastrointestinal (GI) tract. In particular, the present invention discloses methods to extend battery life so that the capsule cameras operate at a lower peak current and/or at a lower frame rate while capable of high frame rate when needed.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot easily reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage, where the capsule camera is in the human gastrointestinal (GI) tract 100. The capsule system 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. System 110 includes battery power supply 24 and an output port 26. Capsule system 110 may be propelled through the GI tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine all images and correctly identify all anomalies. Furthermore, it is desirable to gather location information of the anomalies, which is useful for possible operations or treatment of the anomalies. While various location detection devices could be embedded or attached to the capsule device, it is desirable to develop methods for determining the travelled distance based on images captured.

For capsule cameras, the entire GI examination process is powered by the internal battery. It may take more than 20 hours from the moment that the capsule camera is swallowed to the moment it is excreted from the human body. During the examination process, thousands of images will be captured, processed, and then either be stored on-board or wirelessly transmitted to an external receiver. Due to the small capsule size, the capsule device can only afford very limited power capacity. Therefore, it is very critical to utilize the battery capacity wisely in order to optimize the battery capacity usage.

For capsule endoscope application, cell batteries or button batteries are often used. There are different types of materials (i.e., chemical compositions) used for the batteries, such as alkaline, lithium and silver oxide. For each battery, there is a nominal capacity specified and the capacity is often quoted in milliamp hour (mAh). For example, an SR927 battery may have a capacity of 60 mAh at 1.2V cutoff. The battery has a nominal voltage of 1.55V and is intended for devices operated at about 1.5V. While the battery is designed to maintain relatively constant output voltage, however, the output voltage drops substantially when the battery becomes depleted. Therefore, the nominal capacity of the battery may not be fully utilized as specified. One important factor influencing the usable capacity is the battery load. In general, a load that draws a higher drain current will have lower usable capacity due to various reasons such as internal resistance, polarization effect and/or undesirable chemical reactions inside the battery.

FIG. 2 illustrates an example of battery output voltage vs. discharge capacity for two different drain currents. The drawing is intended to illustrate the nature of usable capacity or discharge capacity at different drain currents (I_{A} and I_{B}). In FIG. 2, curves 210 and 220 correspond to the output voltage vs. discharge capacity at drain currents I_{A} and I_{B} respectively, where I_{A}>I_{B}. When the output voltage drops below a certain level (cutoff voltage 230), the battery may not be able to provide the voltage for the connected device to operate properly. As shown in FIG. 2, at a higher drain current I_{A}, the battery will deliver a lower usable capacity Cap_{A}. On the other hand, at a lower drain current Is, the battery will deliver a higher usable capacity Cap_{B}.

The present invention discloses methods to extend battery life for a device powered by batteries. While the capsule endoscope is illustrated as a such device, the present invention can be used in other electronic devices powered by batteries.

BRIEF SUMMARY OF THE INVENTION

A method and a capsule endoscope incorporating the method are disclosed. According to the method, a peak current in a current profile consumed by the capsule endoscope is identified, wherein the peak current is contributed by at least two sub-tasks associated with operations of the capsule endoscope and said at least two sub-tasks are performed overlapping in time. A running voltage indicating a battery output voltage at or near time instances of the peak current is determined. When the running voltage triggers a condition caused by IR (battery internal-resistance) voltage drop, the overlapping of the said at least two sub-tasks is reduced so as to reduce the peak current.

In one embodiment, the second peak current is low enough so that (the second peak current x battery internal resistance) is low enough to allow said at least two sub-tasks continue to operate with reduced overlapping.

In one embodiment, the condition corresponds to a Power-on-Reset signal triggered by the running voltage being below a threshold. In another embodiment, the condition corresponds to the running voltage being below a threshold.

In one embodiment, said at least two sub-tasks comprise image sensing, image processing, and pre-charging LED light source. In one embodiment, said at least two sub-tasks further comprise image write to an archive memory. In another embodiment, said at least two sub-tasks further comprise image transmission to an external wireless receiver.

In one embodiment, said at least two sub-tasks are spread so that an overlap between two of said at least two sub-tasks is reduced. In one embodiment, said at least two sub-tasks are spread so that an overlap between two of said at least two sub-tasks is reduced to 0.

In one embodiment, said at least two sub-tasks are spread so that a duration for at least one sub-task is extended. In another embodiment, said at least two sub-tasks are spread so that a duration for one highest-current sub-task is extended.

According to another method, when the running voltage triggers a condition caused by IR (battery internal-resistance) voltage drop, at least one of said one or more sub-tasks is switched to another sub-task or the capsule endoscope is operated at a second clock rate lower than the first clock rate so as to reduce the first peak current to a second peak current to keep the capsule endoscope continuing to function.

In one embodiment, said another sub-task corresponds to a low-voltage function capable of operating at a lower voltage. A voltage regulator can be used to provide the lower voltage.

In one embodiment, the method further comprises detecting whether the capsule endoscope has been excreted from a human body when the battery output voltage is still sufficient, and enabling a wireless function at a consumer band for the capsule endoscope to transmit images stored on-board to an external device upon detecting the capsule endoscope being excreted.

In one embodiment, said detecting whether the capsule endoscope has been excreted from the human body comprises detecting pixels of a target image having substantial intensity with very low lighting or no lighting output from lighting sources of the capsule endoscope, and wherein the target image is captured using a camera of the capsule endoscope.

In one embodiment, said detecting the pixels of the target image is based on a subset of pixels less than all pixels of camera sensor array of the capsule endoscope.

In one embodiment, the subset of pixels spreads across a substantial area of camera sensor array of the capsule endoscope.

In one embodiment, a temperature is also used for aid detecting whether the capsule endoscope has been excreted from the human body.

In one embodiment, the external device corresponds to a specially designed wireless device or a mobile phone. In one embodiment, the special designed wireless device or the mobile phone further transmits the images stored on-board to a PC, or LAN (Local Area Network), or to a destination through a cloud network or other internet media.

In one embodiment, said switching at least one of said one or more sub-tasks to another sub-task corresponds to switching a camera sub-task to a wireless sub-task, and wherein the capsule endoscope is switched into a wait or sleep mode prior to the wireless sub-task if the capsule endoscope has not been excreted from a human body and the condition caused by the IR voltage drop is triggered. In one embodiment, the capsule endoscope is waked up from the sleep mode and starts the wireless sub-task upon detection of capsule excretion.

In one embodiment, when capsule excretion is detected, the wireless sub-task is initiated either in active communication or by woken-up from a sleep mode by an external device. In one embodiment, the camera sub-task is disabled, or switched off, or partially working.

In one embodiment, the capsule endoscope is woken up from the sleep mode using a wake-up circuit, and wherein the wake-up circuit uses an event or a combination of events to detect excretion of the capsule endoscope or detected excretion signal is communicated to the capsule endoscope through another wireless device by a user.

In one embodiment, said switching at least one of said one or more sub-tasks to another sub-task corresponds to switching a camera sub-task to an excretion detection sub-task. In one embodiment, the excretion detection sub-task is further switched to a wireless sub-task to transmit images stored on-board upon capsule excretion detected.

### BRIEF DESCRIPTION OF THE DRAWINGS

FIG. 1 illustrates an exemplary capsule system with on-board storage, where the capsule system includes illuminating system and a camera that includes optical system and image sensor.

FIG. 2 illustrates an example of battery output voltage vs. discharge capacity for two different drain currents.

FIG. 3A illustrates an example of a capsule system having a power controller that switches on either the wireless function or the camera function.

FIG. 3B illustrates an example of a capsule system having a power controller that enables/disables the wireless function or the camera function.

FIG. 3C illustrates an example of a capsule system having an efficient DC-to-DC converter that converts the power from high voltage to low voltage.

FIG. 4A illustrates an example of current profiles for the sub-tasks, where the drawing shows the capsule endoscope is operated at near full duty cycle, and the cycle of a next task starts shortly after a current task is completed.

FIG. 4B illustrates an example of current profiles at a low duty cycle, which corresponds to two task cycles per time unit Tu.

FIG. 4C illustrates an example of total current profile for the sub-tasks in FIG. 4A and FIG. 4B.

FIG. 4D illustrates an example of spreading the sub-tasks to reduce or avoid the overlap between at least two sub-tasks according to one embodiment of the present invention.

FIG. 4E illustrates an example of sub-task peak current profiles for the sub-tasks in FIG. 4D.

FIG. 4F illustrates an example of reducing the peak current by reducing the clock rate according to one embodiment of the present invention.

FIG. 4G illustrates an example of sub-task peak current profiles for the sub-tasks in FIG. 4F.

FIG. 5 illustrates an exemplary flowchart for a capsule endoscope incorporating sub-task spreading to reduce overlap between sub-tasks according to an embodiment of the present invention.

FIG. 6 illustrates an exemplary flowchart for a capsule endoscope to switch a sub-task to another sub-tasks to reduce peak current according to an embodiment of the present invention.

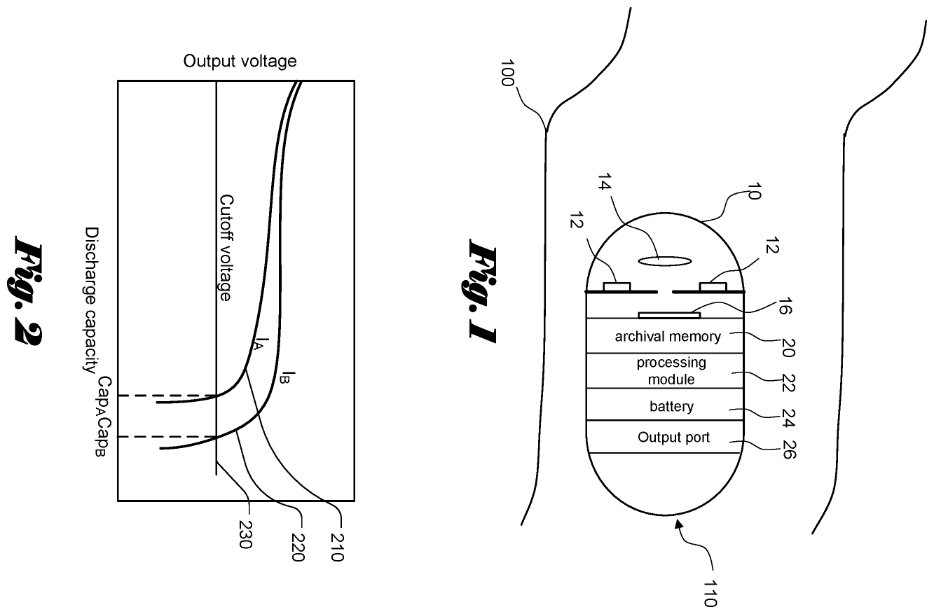

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention. References throughout this specification to “one embodiment,” “an embodiment,” or similar language mean that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

Endoscopes are normally inserted into the human body through a natural opening such as the mouth or anus. Therefore, endoscopes are preferred to be small sizes so as to be minimally invasive. As mentioned before, endoscopes can be used for diagnosis of human gastrointestinal (GI) tract. The captured image sequence can be viewed to identify any possible anomaly. The capsule endoscope may take more than 10 hours to travel through the human GI tract before it is excreted. During the course of travelling through the human GI tract, the capsule endoscope may have to capture tens of thousands images of the GI tract, process the images, and either store images on-board or transmit the images to external receiver. The capsule endoscope has to execute all the related tasks based on the power from one or more tiny batteries inside the capsule housing. Accordingly, the power is a very precious resource to operate the capsule endoscope. Therefore, it is desirable to squeeze out as much power from the batteries as possible. This is also true for many battery-powered electronic devices.

With the advancements in both semiconductors based on Moore's law for electronics with high speed complicated operation consuming low power, and battery technology to increase energy storage in smaller space, come the advent and then proliferation of battery powered devices, for example laptop computer and other handheld devices.

When battery becomes partially depleted, its internal resistance will increase noticeably and the voltage across its terminals will increase that will cause the battery output voltage to drop for the same current. The voltage drop associated with the internal resistance corresponds to (current x battery internal resistance). Therefore, the voltage drop will increase when the battery internal resistance increases for the same current. Consequently, the battery output voltage will drop when the battery becomes partially drained. In this case, the circuits may malfunction. However, though some functions cannot be performed at the same speed, but it still can perform the functions at a different (i.e., lower) speed or change to some other functions that can be performed by consuming a lower current. In one embodiment, the same function can be performed but with a lower speed (e.g. ½ speed) and/or with a lower current. In another embodiment, another function with a lower current can be performed. When the battery is low (i.e., partially to noticeably drained), there may be still some substantial capacity remaining (e.g. 20-30%) before the battery is fully depleted. Embodiments according to present invention can help to squeeze out more battery energy by lowering the peak current consumed so as to extended the capsule operation time.

In another embodiment, the triggering event to switch to a lower speed or different function with a lower current is the battery output voltage dropping below a threshold. Some electronic circuits can be arranged to monitor the battery output voltage. The voltage being monitored (referred as a running voltage in this disclosure) is indicative of the battery output voltage. If the state of battery output voltage below a threshold is triggered, then the system switches to a lower speed or different function requiring a lower current. Another example of triggering event can be a Power-on-Reset signal generated using a PoR circuit. The PoR circuit will hold the reset signal asserted until a time period after the voltage reaches a proper level upon system power on. This will ensure the system can reliably start from a known state. The PoR circuit will also hold rest asserted when the voltage drops below a threshold.

In order to reliably detect the event of battery output voltage below a threshold, the voltage monitoring is performed at or near the instance of peak current. In order to identify the instances of the peak current during the capsule camera operations, an overall current profile is identified for the functions (or sub-tasks) performed by the capsule camera. With the knowledge of the overall current profile associated with functions or sub-tasks, the running voltage can be checked at or near the instances of the peak current. Voltage detection at or near instances around the peak current according to the present invention can trigger the event more reliably. It can detect the voltage issue caused by the IR drop at the earliest moment to avoid possible system malfunction.

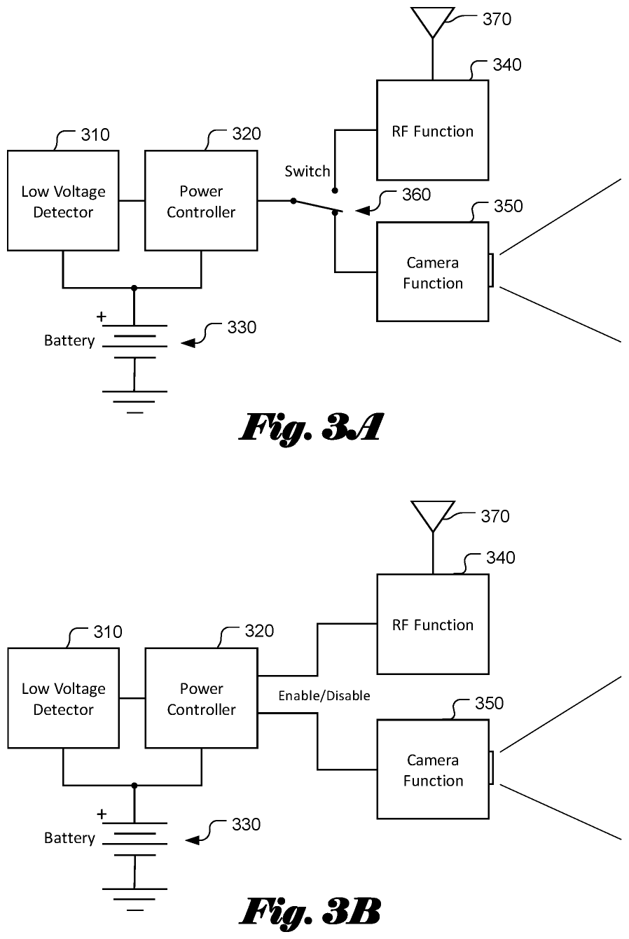

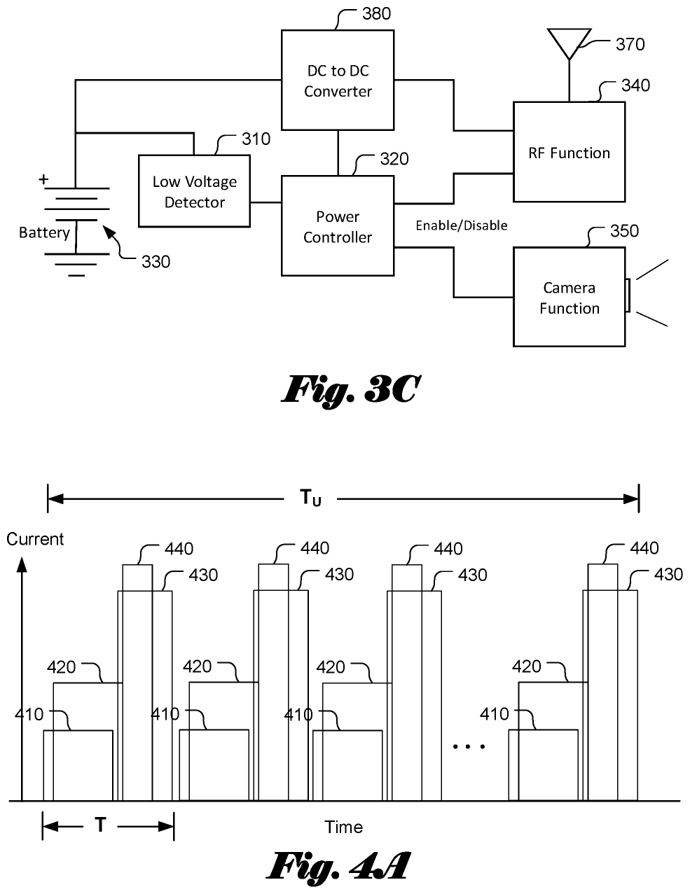

FIG. 3A illustrates an example of a capsule endoscope that switches from one function and another function when a triggering event is detected. The capsule system having a power controller 320 that switches on (via. Switch 360) either the wireless function or RF function 340 or the camera function 350. A low voltage detector 310 is coupled to the battery 330 to detect the low battery event. The low voltage detector 310 can signal the power controller 320 when the low battery event is detected. An antenna 370 is coupled to the RF function 340 for transmit and/or receive. The camera function and the RF function are used to illustrate an example of switching between two functions when an event is triggered. The present invention is not limited to two functions illustrates. Instead, these two functions may comprise other functions such as data read/write from memory, LED flash charging, etc.

In another embodiment, when the battery low event is triggered or detected, some functions are disabled and remaining functions can continue to be performed. In yet another embodiment, different functions are performed with a lower current such that the (peak current x battery internal resistance) is sufficiently low so that the functions are still operating. In yet another embodiment, some lower voltage functions continue to be performed or are activated to be performed since the IR drop (i.e., voltage drop due to battery internal resistance) from the battery internal resistance does not affect the functions with such low-voltage operations. The lower voltage operation may be connected through a voltage regulator. In yet another embodiment, a switching regulator is used to cause less current drawn from the battery to further extend the operation time.

In one embodiment, a lower speed operation can be achieved by using a lower clock speed. In another embodiment, a lower speed operation is achieved through a lower VCC from a regulator output to cause the circuit to function slower, but to draw a less current. In yet another embodiment, the lower current is achieved by a lower analog circuit current setting, such as setting the biasing point with a lower transistor gate voltage, although it might affect the speed or quality.

In one embodiment, the system has a regulator connecting to the battery/batteries; switching to a lower speed may correspond to a lower voltage from the regulator output since the lower speed normally needs a lower VCC in order to further lower the current for the system to last even longer.

In another embodiment, when the switching occurs, the clock rate is lowered, or some modules in the system are shut down or disabled, or its clock is disabled. In another embodiment, a power switch is involved to turn off one function with a higher current and to turn on another function with a lower current. In another embodiment, the lower power function has lower VCC so that a different regular function is involved. With a switching regulator, this lower VCC regulator draws less current from the battery with the original voltage range.

FIG. 3B illustrates an example of a capsule endoscope that uses the power controller 320 to enable/disable one of two functions (340 and 350) for switching from one function to another function when a triggering event is detected.

FIG. 3C illustrates an example of a capsule endoscope that uses the power controller 320 to enable/disable one of two functions (340 and 350) for switching from one function to another function when a triggering event is detected. Furthermore, the capsule endoscope includes a DC-to-DC converter 380 (i.e., a type of switching regulator) to provide a lower voltage for the RF function 340 to reduce the current. In FIGS. 3A-C, RF (i.e., wireless) and camera functions may have a common circuits module or circuits which are not explicitly shown in these figures. In FIG. 3C, there might be circuits or modules with different VCCs communicating with each other.

When the larger than maximum allowable value of the (battery internal resistance×peak current) or below the lowest operation voltage of the battery output voltage is detected, actions have to be taken to disable certain function(s) in order to reduce the current so as to keep the system continue to operate. However, certain other function(s) will only be enabled after a certain time or after a certain event or condition has occurred. For example, the condition corresponds to detection of capsule excreted from the body or detection of battery output voltage below a voltage threshold.

The technique to extend the capsule operation by lowering the system current usage or voltage requirement as mentioned above can be applied to the on-board storage capsule system. While the device travels through the GI tract, the camera takes images and stored them in the on-board storage system. If the capsule is excreted in sufficiently short time, the normal imaging function (i.e., camera function) may still be in operation. The event of capsule exiting the human body can be detected. For example, the pixels of an image having substantial intensity with very low lighting or no lighting output (as measured by the current of the LED light source of the capsule) may indicate that the capsule has excreted from the body. This condition may trigger the capsule to switch from the imaging function to the wireless image transmission function to transmit on-board stored images and/or other data to an external device via a consumer band. The external device, for example, may be a specially designed wireless device or a mobile phone. In one embodiment, a temperature sensor can be included to work with the camera system to detect the excretion of the capsule and to initiate or switch to the wireless function in the capsule. In one embodiment, the special designed wireless device or mobile phone can further transmit the images to a PC or through the cloud, or LAN (Local Area Network), or other internet media to a destination.

If the capsule takes a long time to excrete and the battery may no longer be able to sustain the current due to the increased internal resistance. The power-on-reset function or other circuits, that are capable of detecting the battery or power bus voltage being too low, can be used to trigger the capsule system to switch into a wait or sleep mode of the wireless function. After wakeup, the capsule needs to operate at a lower current than the imaging function, or needs to operate at a lower voltage such that the desired function can be maintained even at the peak current. A temperature sensor can sense the change of the environment and switch the wireless function mode from wait to wake-up mode to transmit the images to a special designed wireless device or a consumer mobile device. After detecting the low battery energy, some camera functions may still operate to detect the capsule excretion. It may operate a low frame rate, without storing the images to avoid the need of high flash memory writing current, or only a subset of pixels from the sensor is used to detect the capsule excretion. In one embodiment the subset is formed by pixels substantially across the sensor array area for better detection of excretion. In one embodiment, the wireless function can be in the wait state to be woken up by manually or according to some pre-defined conditions.

In one embodiment, after the capsule has initially been turned on and operated in the camera function for a certain amount of time, T until the battery internal resistance increases to a certain level, the power-on-reset circuits or other circuits that are capable of detecting the battery or power bus voltage being too low can trigger the capsule system to switch from the camera function to the wireless function. The triggering point can be set based on the case requiring the peak operation current. Upon triggering, the system can be placed initially in a wait, sleep or wakeup mode, or initiate communication with an external device, such as a mobile phone. Alternatively, the wireless function can be turned on, whether in active communication state or in wait state to be woken up by an external device, at the excretion of the capsule. In one embodiment when the low voltage threshold at the peak current is detected, the camera enters a low-throughput monitor mode with low frame rate or a mode where only a subset of pixels are sensed with no light by the capsule LED's to detect the excretion of the capsule.

In another embodiment, a temperature sensor or other sensor can be used to cause the wireless function to change from the wait to the wake-up mode to start transmitting image data. A protocol can be executed between the capsule wireless system and the other wireless system, such as a smart phone or other wireless device, to establish the communication and enable the transfer of image data. In one embodiment, the switch from the camera function to the wireless function is through power switch (i.e., switching the power on/off for the circuits responsible for the camera function or the wireless function). In another embodiment, the wireless device may have a lower VCC requiring a different regulator. In another embodiment, this regulator is a switching regulator. In another embodiment, the wireless function is enabled from a disabled state.

In one embodiment, the wake-up circuit may use an event or a combination of events to detect the excretion of the capsule, or the detected excretion signal can be communicated to the capsule through another wireless device by the user, which is normally a patient. In another example, the wake-up circuit is disabled until the excretion of the capsule and the capsule can start to hear any signal from another wireless device. In yet another example, the capsule also can be the initiator of the wireless communication after the excretion is detected. In one embodiment, the duty cycle of the capsule to initiate the communication is small and the image transmission can start after handshake is made.

In another embodiment, after a certain camera operation time, T, the battery internal resistance increases to a level to trigger the power-on-reset or other circuits that are capable of detecting the battery or power bus voltage being too low, the camera function is disabled. A temperature sensor can be used to monitor the temperature change in the environment after excretion in order to initiate the wireless function.

In the above description, the concept of “wait for a period time, T” may increase reliability if the power system bounces. However, it may not be necessary, for example, especially when the system is robust in terms of contact bounce.

In the above discussion, when switching to a lower current mode, the lower current can be achieved by rescheduling the events within the function being performed by detaching the current consuming events so that the peak current causing the highest voltage drop through the battery resistance becomes lower.

All the switching methods mentioned above may be affected by the power-on-reset or some circuits detecting the voltage between VCC and GND being below a certain threshold. It also can be switched after a certain time TS, some accumulated results, such as the number of instructions executed, the number of images taken, the (total current×time) (i.e. charges) from the battery, the total power from the battery, the total number of pixels stored, or a combination thereof.

In one embodiment, after time TS or a number of images taken, or a combination thereof, the events may be scheduled differently so that one or more events are detached to reduce the peak current, which increases the total battery life or to increase the number of images taken. This may reduce the possible peak frame rate, Nevertheless, at the end of gastrointestinal tract, the capsule usually travels in a lower speed, which requires a lower frame rate for adequate examination. Also, the issue of prolonged operation time and the higher total number of images taken become a more important measure for complete study (i.e., the capsule still functioning when existing the gastrointestinal tract or reaching some anatomy that the procedure is considered complete).

In another embodiment, after time, TS or a number of images taken, or a combination thereof, the clock may be reduced. When the power-on-reset or other circuits that are capable of detecting the battery or power bus voltage being too low is triggered, the system is switched to sleep, wait state or wireless transmission. In the sleep or wait state, some events (e.g., detecting the capsule exiting form the human body) may trigger the wireless operation. In yet another embodiment, the switch into a next lower voltage current function may be put on hold to be executed after the detection of a certain event.

The wireless function sometimes can be trigger in vivo however. Since FCC requires in vivo medical device to operate at a medical implant band, and the capsule can be operated in a consumer band or another band different from the medical implant band once the capsule is excreted. In this embodiment, the wireless system has a dual band function and an antenna, and the antennas is capable of transmitting in dual band frequencies or two antennas are used, one for each band.

The capsule endoscope moves slowly in the GI tract. Due to the slow movement, the capsule endoscope usually captures images at a very slow frame rate, such as 2-5 frames per second. The current image sensor technology and the circuit technology are capable of capturing and processing images at a much higher frame rate, such as 10 frames per second. Accordingly, the capsule endoscope is usually operated at a relatively low duty cycle.

Let's assume that one task will take duration T to complete, which results in 1/T repetitions per second for the task. For example, T corresponds to 0.1 second, which results in a maximum throughput rate of 10 repetitions of the task per second. In the case of a capsule endoscope with on-board storage, each task comprises capturing the image, processing the image and storing the image on-board. In this example, the capsule endoscope is capable of handling 10 frames per second. If we take 3 frames per second, then the capsule endoscope is id1e70% of the time. In other words, the “duty cycle” is 30%.

In a capsule endoscope, each image capture (either recorded or transmitted) can be considered as a task. The sub-activities of image capture task in terms of current may comprise image sensing by the image sensor, image processing by the processor, flash memory writing, and lighting energy (e.g., LED light) pre-charge for the subsequent image sensing, with some of them overlapped in time. These sub-tasks are just an example to illustrates the incorporating the present invention in a capsule endoscope. For other battery powered devices, different sub-tasks may be identified for the target task. These sub-tasks are often performed with some overlap. For example, the image sensing sub-task will output image data on a line-by-line basis. Image processing, such as de-mosaicking and image compression may start upon sufficient image data (e.g., 8 lines) collected. The flash memory write can start after sufficient image data are processed. On the other hand, the LED capacitor can be pre-charged after the current image is sensed so that the LED light can be ready to trig for the next frame when needed.

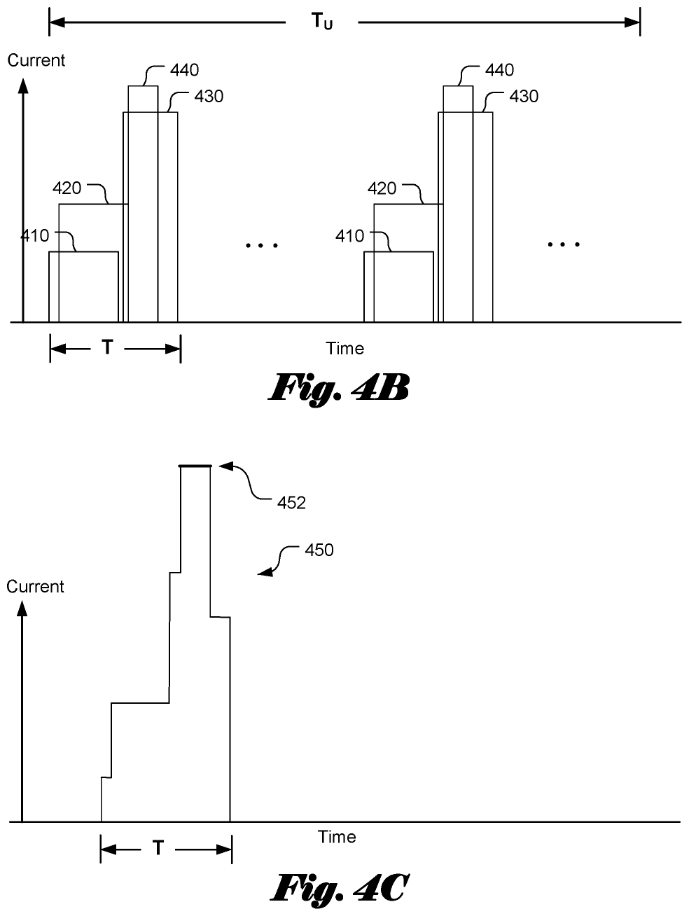

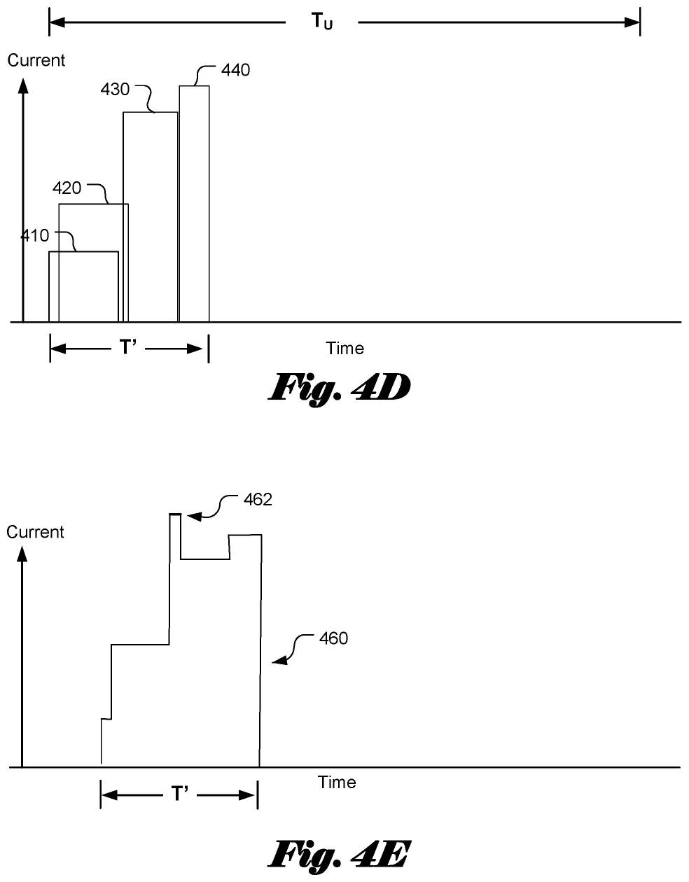

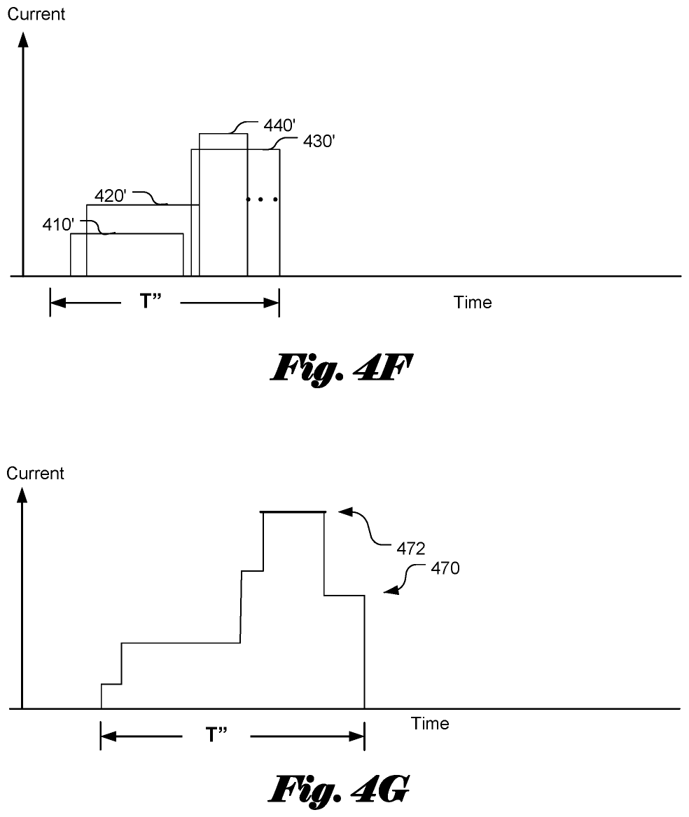

FIG. 4A illustrates an example of current profiles for the sub-tasks, where the drawing shows the capsule endoscope is operated at near full duty cycle, and the cycle of a next task starts shortly after a current task is completed. The duration for each cycle is T and there are T_{U}/T tasks that can be executed during a time unit Tu. The time unit can be any specified period, such as 1 second. The higher frame rate sometimes is needed at a certain part of GI tract, such as esophagus, or when fast motion is detected that triggers a high frame rate.

As mentioned earlier, when the becomes depleted, the battery internal resistance increases and the voltage drop associated with the internal resistance will increase for a same current. In order to extend the capsule operation, the capsule endoscope can be operated at a lower duty cycles according to one embodiment. In this disclosure, the term “battery depleted” refers to the situation that a portion of the battery energy has been consumed and the internal resistance has been increased so that the battery output voltage is noticeably dropped. The lower voltage output from the battery may affect the capsule system powered by the battery. While the battery is depleted (not fully depleted), there is still substantial remaining battery energy. FIG. 4B illustrates an example to lower the frame rate to two frames per time unit T_{U}. Therefore, there is a long idle time between two tasks.

The major tasks illustrated in the example of FIG. 4A correspond to image sensing 410, image processing 420, flash memory write 430 and LED capacitor pre-charge for the next sensing 440. In FIG. 4A, the current profiles of the sub-activities are shown as box shapes for the illustration purpose. In practice, the current profile may be smooth curves having a rise time and a fall time due to non-zero transistor turn-on time and capacitance exiting in the load circuits between VCC and GND. When two sub-tasks overlap, the total current adds up. For example, the image sensing 410 and image processing 420 have substantial overlap and the total current corresponds to the sum of two individual currents during the overlapped period. Also, the LED capacitor pre-charge for the next sensing sub-task 440 fully overlaps with the flash memory write sub-task 430. Therefore, the total current during the LED capacitor pre-charge time corresponds to the sum of currents for the LED capacitor pre-charge and the flash memory write. The total current profile 450 of the sub-tasks is shown in FIG. 4C. The peak current 452 occurs during the overlapped period of the LED capacitor pre-charge and the flash memory write. In FIG. 4C, the vertical scale is compressed compared to that of FIG. 4A and FIG. 4B in order to properly fit the drawing within the page.

In one embodiment of the present invention, the capsule endoscope can be configured to control when to start individual sub-tasks so as to reduce the overlap among the sub-tasks.

When the battery becomes partially or noticeably depleted, the peak current 452 as shown in FIG. 4C may cause significant voltage drop at the battery output voltage due to increase of the battery internal resistance and consequently cause the capsule fail to operate properly. While the approach shown in FIG. 4B to lower the frame rate (while maintaining the frame period) may help to ease the current drainage to some degree, the current profile for each frame capture stays the same as shown in FIG. 4C. According to another embodiment of the present invention, the capsule endoscope may spread certain peak-current causing sub-tasks when an event indicating the battery being depleted is triggered. For example, triggering event may correspond to PoR due to battery voltage below a voltage threshold. For this technique to work effectively, the sub-tasks that cause the peak current need to be identified. For example, the peak current 452 in FIG. 4C is contributed by sub-tasks 430 and 440. While sub-tasks 410 and 420 are also overlapped, spreading sub-tasks 410 and 420 won't help to lower the peak current. FIG. 4D illustrates an example of sub-task spreading according to one embodiment of the present invention, where sub-tasks 430 and 440 are spread by delaying the start time for task 440 so that it doesn't overlap with sub-task 430. In other words, LED capacitor pre-charge (i.e., sub-task 440) is delayed until the flash memory write (i.e., sub-task 430) is completed. In this example, the frame capture time T′ becomes longer than before. The total current profile 460 is shown in FIG. 4E, where the new peak current 462 corresponds to the overlap between tasks 420 and 430, which is much lower than the original peak current 452 in FIG. 4C.

In certain cases, some sub-tasks are interchangeable. For example, in the above case, the writing to flash memory and pre-charging the capacitor to store energy for LED lighting for the next frame are interchangeable, as long as the peak current is reduced by spreading out the sub-tasks. The present invention also covers variations of sub-task spreading, such as reversing the sequence order of the sub-tasks or changing the sequence order if the orders in the sequence are interchangeable.

As is known in the field, a battery at a higher drain current will provide less total energy than it will at a lower drain current. Therefore, embodiments of the present invention to spread the sub-tasks to reduce or avoid any overlap between two sub-tasks will allow the capsule endoscope to derive more electrical capacity from the batteries. Beside spreading the sub-tasks to reduce or avoid overlap, individual sub-tasks may also be spread as long as the system has sufficient time to complete the task. For example, the LED may take a longer duration to pre-charge so as to reduce the charging current. In another example, both LED pre-charge and memory write sub-tasks may spread the duration so that both sub-tasks will result in reduced peak current.

For digital part of the functions with lower performance requirements (e.g., lower frame rate), lowering the clock can lower the peak current. While this is one way to lower the peak current, there are other ways to reduce or avoid the parallel processing of sub-tasks when possible, but still meet the performance requirement. This can be realized by one or more control circuits. It may also be flexibly realized by a CPU or a multiple-core system, or a combination of CPU, one or more processors, and one or more circuits. For example, a smartphone may be performing MPEG video compression while performs facial recognition in real time on each image. At a fast frame rate, substantial overlap of these two functions or sub-tasks may be required. However, at a lower frame rate, the overlap between these two functions or sub-tasks may be reduced or avoided.

In yet another embodiment, the sub-task spreading is achieved by performing some selected sub-tasks over longer periods of time. For example, the sub-task spreading can be achieved by slowing down the operating clock so that the duration for a target sub-task is increased by a factor depending on the ratio of the original clock and the slowed-down clock. In this case, the target sub-task is spread so that the consumed current by the target sub-task is reduced over a longer period. Similar to the method of slowing down the clock, if a target sub-task is implemented using one or more processors, CPUs or ASIC, said one or more processors, CPUs or ASIC can be programmed to take longer time to finish the target sub-task. For example, AISC may be used to implement image compression (i.e., part of image processing) and the ASIC can be configured to take twice as long to finish the target sub-task in order to reduce the peak current associated with the target sub-task. In another example, the target sub-task corresponds to the LED pre-charge current, where a capacitor is often used to hold the charges for triggering the LED light. In this case, we could lower the pre-charge current by charging it over a longer period of time. The pre-charge current may be controlled by a CPU via some current control circuits.

FIG. 4F illustrates an example of lowering the peak current by lowering the system clock. If the sub-tasks in FIG. 4B correspond to sub-tasks performed by digital circuits, the current usage will be reduced when the clock rate is reduced. The corresponding currents for 410-440 become currents 410′-440′. The overall current profile 470 is shown in FIG. 4G, where the peak current 472 is reduced from the current 452 in FIG. 4C.

In some cases, the system time may be constrained so that sub-tasks cannot be fully spread to avoid overlap between two sub-tasks. However, as long as the overlap duration is reduced, the duration of high peak current is reduced and it helps to derive higher battery capacity.

In the above discussion, the capsule endoscope is used to illustrate the method of extending battery life by spreading the sub-tasks to lower the peak current. However, the present invention is not limited to the capsule endoscope. Any battery powered device, such as IoT (Internet of Thing) devices, wearable devices or smart phones can be benefitted from the present invention by adopting sub-task spreading.

The method mentioned above can be implemented using various programmable devices such as micro-controller, central processing unit (CPU), field programmable gate array (FPGA), digital signal processor (DSP), ASIC (Application Specific Integrated Circuit) or any programmable processor or circuitry.

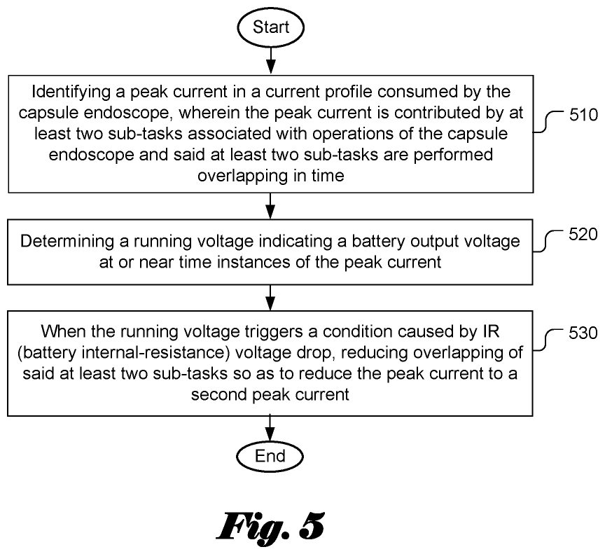

FIG. 5 illustrates another exemplary flowchart for a capsule endoscope to reduce overlapping between sub-tasks according to an embodiment of the present invention. According to this method, a peak current in a current profile consumed by the capsule endoscope is identified in step 510, wherein the peak current is contributed by at least two sub-tasks associated with operations of the capsule endoscope and said at least two sub-tasks are performed overlapping in time. A running voltage indicating a battery output voltage at or near time instances of the peak current is determined in step 520. When the running voltage a condition caused by IR (battery internal-resistance) voltage drop, said at least two sub-tasks are adjusted to reduce overlapping so as to reduce the peak current to a second peak current in step 530.

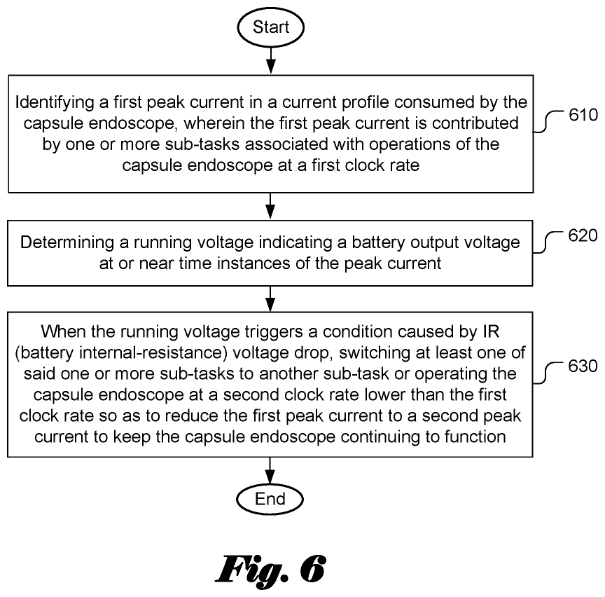

FIG. 6 illustrates another exemplary flowchart for a capsule endoscope to reduce clock rate or switch sub-tasks according to an embodiment of the present invention. According to this method, a first peak current in a current profile consumed by the capsule endoscope is identified in step 610, wherein the first peak current is contributed by one or more sub-tasks associated with operations of the capsule endoscope at a first clock rate. A running voltage indicating a battery output voltage at or near time instances of the first peak current is determined in step 620. When the running voltage triggers a condition caused by IR (battery internal-resistance) voltage drop, at least one of said one or more sub-tasks is switched to another sub-task or the capsule endoscope is operated at a second clock rate lower than the first clock rate so as to reduce the first peak current to a second peak current to keep the capsule endoscope continuing to function in step 630.

The above description is presented to enable a person of ordinary skill in the art to practice the present invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the above detailed description, various specific details are illustrated in order to provide a thorough understanding of the present invention. Nevertheless, it will be understood by those skilled in the art that the present invention may be practiced.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of extending battery life for a capsule endoscope powered by a battery, the method comprising:
identifying a peak current in a current profile consumed by the capsule endoscope, wherein the peak current is contributed by at least two sub-tasks associated with operations of the capsule endoscope and said at least two sub-tasks are performed overlapping in time;
determining a running voltage indicating a battery output voltage at or near time instances of the peak current; and
when the running voltage triggers a condition caused by IR (battery internal-resistance) voltage drop, reducing overlapping of said at least two sub-tasks so as to reduce the peak current to a second peak current.

2. The method of claim 1, wherein the second peak current is low enough so that (the second peak current x battery internal resistance) is low enough to allow said at least two sub-tasks continue to operate with reduced overlapping.

3. The method of claim 1, wherein the condition corresponds to a Power-on-Reset signal triggered by the running voltage being below a threshold.

4. The method of claim 1, wherein the condition corresponds to the running voltage being below a threshold.

5. The method of claim 1, wherein said at least two sub-tasks comprise image sensing, image processing, and pre-charging LED light source.

6. The method of claim 5, wherein said at least two sub-tasks comprise image write to an archive memory.

7. The method of claim 5, wherein said at least two sub-tasks comprise image transmission to an external wireless receiver.

8. The method of claim 1, wherein said at least two sub-tasks are spread so that an overlap between two of said at least two sub-tasks is reduced.

9. The method of claim 1, wherein said at least two sub-tasks are spread so that an overlap between two of said at least two sub-tasks is reduced to 0.

10. The method of claim 1, wherein said at least two sub-tasks are spread so that a duration for at least one sub-task is extended.

11. The method of claim 1, wherein said at least two sub-tasks are spread so that a duration for one highest-current sub-task is extended.

12. A capsule endoscope, comprising:
a pixel array being responsive to light energy received by the pixel array;
an LED light source to illuminate a scene for the pixel array;
one or more circuits coupled to the pixel array and the LED light source; and
a battery to supply electrical power to the pixel array, the LED light source and said one or more circuits; and
a housing adapted to be swallowed, wherein the battery, the pixel array, the LED light source and said one or more circuits are enclosed in the housing; and
wherein said one or more circuits, the pixel array and the LED light source are configured to:
monitor a peak current in a current profile consumed by the capsule endoscope, wherein the peak current is contributed by at least two sub-tasks associated with operations of the capsule endoscope and said at least two sub-tasks are performed overlapping in time;
determine a running voltage indicating a battery output voltage at or near time instances of the peak current; and
when the running voltage triggers a condition caused by IR (battery internal-resistance) voltage drop, reduce overlapping of said at least two sub-tasks so as to reduce the peak current to a second peak current.

13. A method of leveraging battery energy for a capsule endoscope powered by a battery, the method comprising:
identifying a first peak current in a current profile consumed by the capsule endoscope, wherein the first peak current is contributed by one or more sub-tasks associated with operations of the capsule endoscope at a first clock rate;
determining a running voltage indicating a battery output voltage at or near time instances of the first peak current; and
when the running voltage triggers a condition caused by IR (battery internal-resistance) voltage drop, switching at least one of said one or more sub-tasks to another sub-task or operating the capsule endoscope at a second clock rate lower than the first clock rate so as to reduce the first peak current to a second peak current to keep the capsule endoscope continuing to function.

14. The method of claim 13, wherein said another sub-task corresponds to a low-voltage function capable of operating at a lower voltage.

15. The method of claim 14, wherein a voltage regulator is used to provide the lower voltage.

16. The method of claim 13, wherein the condition corresponds to a Power-on-Reset signal triggered by the running voltage being below a threshold.

17. The method of claim 13, wherein the condition corresponds to the running voltage being below a threshold.

18. The method of claim 13 further comprises detecting whether the capsule endoscope has been excreted from a human body when the battery output voltage is still sufficient, and enabling a wireless function at a consumer band for the capsule endoscope to transmit images stored on-board to an external device upon detecting the capsule endoscope being excreted.

19. The method of claim 18, wherein said detecting whether the capsule endoscope has been excreted from the human body comprises detecting pixels of a target image having substantial intensity with very low lighting or no lighting output from lighting sources of the capsule endoscope, and wherein the target image is captured using a camera of the capsule endoscope.

20. The method of claim 19, wherein said detecting the pixels of the target image is based on a subset of pixels less than all pixels of camera sensor array of the capsule endoscope.

21. The method of claim 20, wherein the subset of pixels spreads across a substantial area of camera sensor array of the capsule endoscope.

22. The method of claim 19, wherein a temperature is also used for aid detecting whether the capsule endoscope has been excreted from the human body.

23. The method of claim 18, wherein the external device corresponds to a specially designed wireless device or a mobile phone.

24. The method of claim 23, wherein the special designed wireless device or the mobile phone further transmits the images stored on-board to a PC, or LAN (Local Area Network), or to a destination through a cloud network or other internet media.

25. The method of claim 13, wherein said switching at least one of said one or more sub-tasks to another sub-task corresponds to switching a camera sub-task to a wireless sub-task, and wherein the capsule endoscope is switched into a wait or sleep mode prior to the wireless sub-task if the capsule endoscope has not been excreted from a human body and the condition caused by the IR voltage drop is triggered.

26. The method of claim 25, wherein the capsule endoscope is waked up from the sleep mode and starts the wireless sub-task upon detection of capsule excretion.

27. The method of claim 25, wherein when capsule excretion is detected, the wireless sub-task is initiated either in active communication or by waken-up from a sleep mode by an external device.

28. The method of claim 27, wherein the camera sub-task is disabled, or switched off, or partially working.

29. The method of claim 25, wherein the capsule endoscope is waken up from the sleep mode using a wake-up circuit, and wherein the wake-up circuit uses an event or a combination of events to detect excretion of the capsule endoscope or detected excretion signal is communicated to the capsule endoscope through another wireless device by a user.

30. The method of claim 13, wherein said switching at least one of said one or more sub-tasks to another sub-task corresponds to switching a camera sub-task to an excretion detection sub-task.

31. The method of claim 30, wherein the excretion detection sub-task is further switched to a wireless sub-task to transmit images stored on-board upon capsule excretion detected.

32. A capsule endoscope, comprising:
a pixel array being responsive to light energy received by the pixel array;
an LED light source to illuminate a scene for the pixel array;
one or more circuits coupled to the pixel array and the LED light source; and
a battery to supply electrical power to the pixel array, the LED light source and said one or more circuits; and
a housing adapted to be swallowed, wherein the battery, the pixel array, the LED light source and said one or more circuits are enclosed in the housing; and
wherein said one or more circuits, the pixel array and the LED light source are configured to:
monitor a first peak current in a current profile consumed by the capsule endoscope, wherein the first peak current is contributed by one or more sub-tasks associated with operations of the capsule endoscope at a first clock rate;
determine a running voltage indicating a battery output voltage at or near time instances of the first peak current; and
when the running voltage triggers a condition caused by IR (battery internal-resistance) voltage drop, switch one of said one or more sub-tasks to another sub-task or operate the capsule endoscope at a second clock rate lower than the first clock rate so as to reduce the first peak current to a second peak current to keep the capsule endoscope continuing to function.
